(19) United States
(12) Patent Application Publication
(10) Pub. No.: US 2012/0277448 A1
(43) Pub. Date: Nov. 1, 2012

(54) PREPARATION METHOD FOR 3BETA-ARACHIDYLAMIDO-7ALPHA, 12ALPHA, 5BETA-CHOLAN-24-CARBOXYLIC ACID

(75) Inventors: Xiangrui Jiang, Shanghai (CN); Lijun Guo, Shanghai (CN); Yiru Dai, Shanghai (CN); Jianfeng Li, Shanghai (CN); Jin Suo, Shanghai (CN); Rongxia Zhang, Shanghai (CN); Zheng Liu, Shanghai (CN)

(73) Assignees: Shanghai Institute of Materia Medica, Chinese Academy of Sciences, Shanghai (CN); TOPHARMAN SHANGHAI CO., LTD., Shanghai (CN)

(21) Appl. No.: 13/520,117

(22) PCT Filed: Nov. 25, 2010
(86) PCT No.: PCT/CN10/79143

§ 371 Date: Jun. 29, 2012

(30) Foreign Application Priority Data
Dec. 30, 2009 (CN) 200910247675.5

(51) Int. Cl. C07J 41/00; C07J 9/00

(52) U.S. Cl. 552/521; 552/553

(57) ABSTRACT

A method for preparing 3β-arachidylamido-7α,12α,5β-cholan-24-carboxylic acid represented by the following formula V is disclosed, which includes the following steps: converting cholic acid to the compound of formula III by acylation reaction and azidation reaction, reducing the compound of formula III to the compound of formula IV and in the end acylating the compound of formula IV with arachidoyl chloride to get the compound of formula V. The method avoids the use of protection groups.

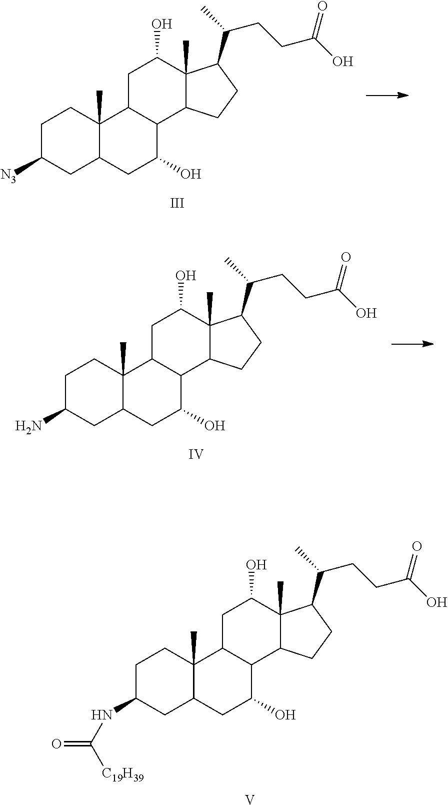

### FIELD OF THE INVENTION

The present invention relates to a method for preparing cholic acid derivatives, specially, relates to a method of preparing 3β-arachidylamido-7α, 12α,5β-cholan-24-carboxylic acid.

### BACKGROUND OF THE INVENTION

3β-arachidylamido-7α,12α,5β-cholan-24-carboxylic acid (Aramchol) is a new compound for treating cholelithiasis which can delay the crystalline growth velocity of cholesterol and promote the dissolving of formed cholesterol crystal (Gut, 2001, 48, 75-79; Lipids, 2001, 36, 1135-1140; Hepatology, 35, 597-600; WO 1999052932; WO 2009060452). Besides cholelithiasis, the compound can be used for treating fatty liver caused by bad eating habit, decreasing the probability of arteriosclerosis, and increasing the secretion of neutral stearyl alcohol (Hepatology, 38, 436-442; Pathobiology 2002, 70, 215-218; Biochem. Soc. Trans., 2004, 32, 131-133). The said compound has a structural formula as follows:

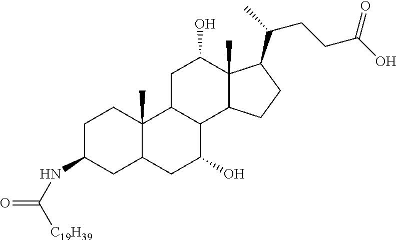

At present, 3β-arachidylamido-7α,12α,5β-cholan-24-carboxylic acid is mainly synthesized by the following method.

For a start, compound 4 is obtained by using natural product of cholic acid as a raw material through steps of reactions in total, i.e. methyl-esterification, p-tosylation, azidation and reduction (Tetrahedron, 26, 2006, 11178-11186; Tetrahedron, 26, 2006, 6808-6813). Generally, the end produce Aramchol is then obtained by acylating and hydrolyzating compound 4. The synthesis steps are as follows:

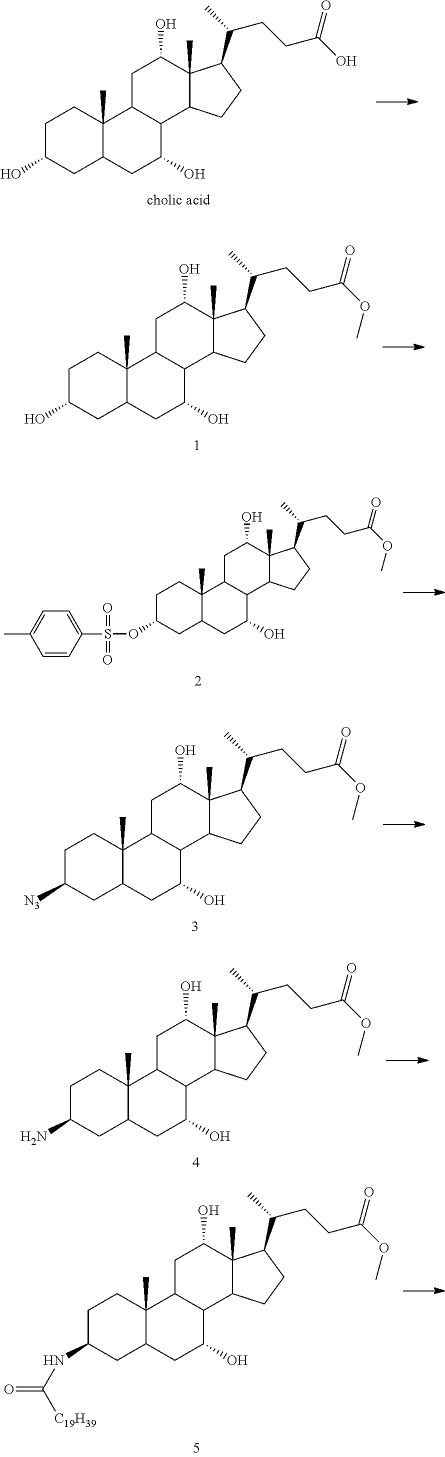

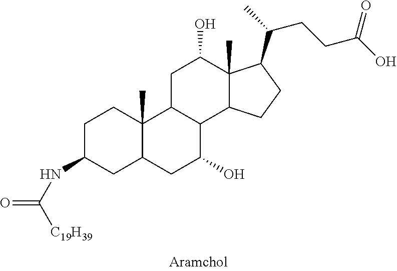

The above synthesis method including 6 steps is too long, wherein the carboxyl must be transferred into a methyl ester group and then hydrolyzed. The use of protecting group of methyl results in two additional steps, i.e., introduction and split of the protecting group, which leads to additional synthesis steps, decreasing overall yield, consuming more raw materials and reagents, aggravating environmental pollution, affecting production efficiency, and thus being bound to cause the increase of synthesis cost.

### SUMMARY OF THE INVENTION

The present invention provides a new method for synthesizing 3β-arachidylamido-7α,12α,5β-cholan-24-carboxylic acid (Aramchol) by a reaction path comprising two steps. The new method avoids using the protection group, decreases reaction steps, lowers unwanted raw material consumption, improves production efficiency, decreases environmental pollution, and has advantages of high yield, low cost and short preparation period.

Thus, an object of the present invention is to provide a method for preparing 3β-arachidylamido-7α,12α,5β-cholan-24-carboxylic acid (Aramchol).

According to the present invention, 3β-arachidylamido-7α,12α,5β-cholan-24-carboxylic acid (Aramchol) can be prepared by a method as follows (Scheme 1).

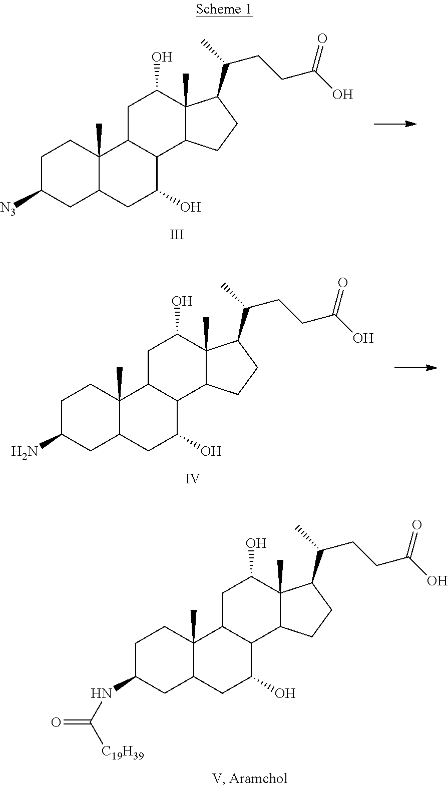

(1) preparing Compound IV by reducing Compound III;

(2) preparing Compound V (i.e. 3β-arachidylamido-7α,12α,5β-cholan-24-carboxylic acid, Aramchol) by acylation of Compound IV and arachidyl chloride in the presence of a base.

In the above method,

in the step (1), azide group in the structure of Compound III is transferred into amino through reduction reaction. The said reduction reaction can be performed in methanol, ethanol, isopropyl alcohol, tetrahydrofuran, dimethoxyethane, 1,4-dioxane, N,N-dimethyl formamide, N,N-dimethyl acetamide, acetonitrile or water, or in a mixture thereof. The catalyst used in the reaction can be, for example, Pd—C, active nickel, cobalt chloride, nickel chloride, iron chloride or the like; the reductant used in the reaction can be, for example, hydrogen gas, sodium borohydride, potassium borohydride, lithium borohydride, hydrogen sulfide, triphenylphosphine or the like; the reaction temperature is not limited, and can be changed within a wide range, and in general from −20° C. to 100° C., preferably, 0° C. to 80° C.

In the step (2), the acylation of Compound IV and arachidyl chloride can be performed in an inert solvent; the used inert solvent can be, for example, dichloromethane, chloroform, 1,2-dichloroethane, tetrahydrofuran, 1,4-dioxane, toluene, acetonitrile, ethyl acetate, pyridine, DMF, dimethoxyethane or the like; the said base can be an organic alkali or inorganic alkali, for example, potassium carbonate, sodium carbonate, triethylamine, pyridine, N,N-dimethyl-4-aminopyridine, diisopropylethylamine, imidazole or the like; the reaction temperature is not limited, and can be changed within a wide range, and in general from −40° C. to 100° C., preferably, −20° C. to 40° C.; the mole ratio of Compound IV to arachidyl chloride is 1:0.8 to 1:3, preferably, 1:1 to 1:1.5.

Wherein, Compound III in the above reaction can be prepared by the azidation reaction of Compound II and an aziding agent.

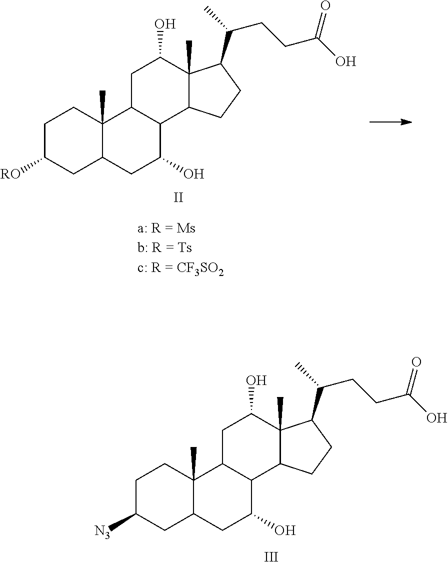

In the presence of the acidic agent, Compound III can be prepared by the azidation reaction of Compound II and an aziding agent; the said aziding agent can be sodium azide, potassium azide, trimethylsilyl azide or the like.

The azidation reaction of Compound II and the aziding agent can be performed in a polar solvent such as N,N-dimethyl formamide, N,N-dimethyl acetamide, acetonitrile, tetrahydrofuran, 1,4-dioxane, toluene, ethyl acetate, dimethoxyethane or the like; the said acidic agent can be an organic acid or inorganic acid, for example, ammonium chloride, ammonium sulphate, ammonium bisulfate or the like; the reaction temperature is not limited, and can be changed within a wide range, and in general from −20° C. to 100° C., preferably, 20° C. to 80° C.

Wherein, Compound II in the above reaction can be prepared from cholic acid (Compound I) through an acylation reaction.

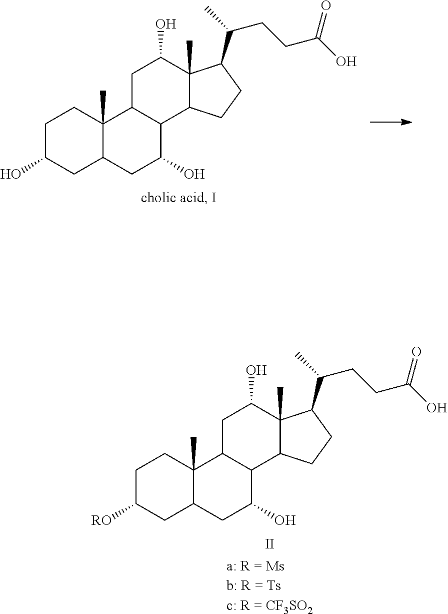

In the presence of a base, Compound II (II-a: R=Ms; II-b: R=Ts; II-c: R=CF₃SO₂) is obtained by the acylation reaction of cholic acid I and RCI; wherein, group R is defined as an easily-removed group, for example, mesyl(Ms), p-tosyl(Ts), trifyl (CF₃SO₂) or the like.

The acylation reaction of cholic acid and p-toluene sulfonyl chloride or methylsulfonyl chloride or trifluoromathanesulfonylchloride can be performed in a non-protonic solvent such as dichloromethane, chloroform, 1,2-dichloroethane, tetrahydrofuran, 1,4-dioxane, toluene, acetonitrile, ethyl acetate, pyridine, DMF, dimethoxyethane or the like; the said base is an organic alkali or inorganic alkali, for example, potassium carbonate, sodium carbonate, triethylamine, pyridine, N,N-dimethyl-4-aminopyridine, diisopropylethylamine, imidazole or the like; the reaction temperature is not limited and can be changed within a wide range, in general from −40° C. to 100° C., preferably, −20° C. to 40° C.; the mole ratio of cholic acid to p-toluene sulfonyl chloride or methylsulfonyl chloride or trifluoromathanesulfonylchloride is 1:0.8 to 1:3, preferably, 1:1 to 1:1.5.

It can be testified from experiments that the method of the present invention has a few reaction steps, a high yield and a moderate reaction condition, and can be easily operated. Furthermore, a product with stable quality and high purity can be obtained by the method according to the present invention. However, as to the synthesis path in the prior arts, carboxyl must be transferred into a methyl ester group which is then hydrolyzed into carboxyl. On the contrary, compared with the synthesis path in the prior arts, two steps of the reactions, i.e. methyl-esterification of carboxyl and hydrolyzation for deprotection, are eliminated from that of the present invention so as to avoid using the protecting group and decrease the reaction steps, which is beneficial to improve overall yield, decrease environmental pollution, enhance production efficiency and decrease unwanted raw material consumption, and may decrease production cost effectively and shorten preparation period. The method has high controllability, and thus can be used for industrial enlargement in commercial scale.

### BEST MODE FOR CARRYING OUT THE INVENTION

All the used agents are analytical reagents (AR) without being further purified; NMR spectrometer is Bruker AMX-400/600, wherein deuterated solvent is CDCl₃, internal standard is TMS; mass spectrometer is Finnigan MAT-95/711.

### Example 1

### Preparation of 3α-methylsulfonyloxy-7α,12α-dihydroxy-5β-cholan-24-carboxylic acid (Compound II-a)

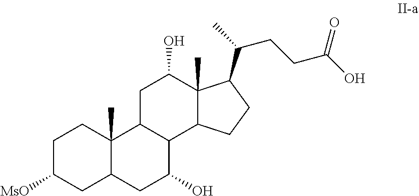

30 g (73.5 mmol) of cholic acid was dissolved in 100 ml of pyridine, cooled to 0° C., and 6.8 ml of methylsulfonyl chloride (88.2 mmol) was then added thereinto. Next, the reaction mixture was continuously stirred for 2 h at 0 to 10° C. and poured into a mixture of 100 ml of ethyl acetate and 100 ml of water with stirring. Kept the mixture standing to separate out an organic layer. The water layer was washed with 100 ml of ethyl acetate for one time. Then the combined organic phase was washed with 50 ml of saturated salt solution, dried and concentrated under reduced pressure to give II-a as a white foam solid which can be used in the next step of reaction directly.

### Example 2

### Preparation of 3α-methylsulfonyloxy-7α,12α-dihydroxy-5β-cholan-24-carboxylic acid (Compound II-a)

30 g (73.5 mmol) of cholic acid was dissolved in 100 ml of pyridine and 100 ml of dichloromethane, cooled to 0° C., and 6.8 ml of methylsulfonyl chloride (88.2 mmol) was then added thereinto. The mixture was continuously stirred for 2h at 0 to 10° C. and poured into a mixture of 100 ml of ethyl acetate and 100 ml of water with stirring. Kept the mixture standing to separate out an organic layer. The water layer was washed with 100 ml of ethyl acetate for one time. Then the combined organic phase was washed with 50 ml of saturated salt solution, dried, and concentrated under reduced pressure to afford II-a as a white foam solid which can be used in the next step of reaction directly.

### Example 3

### Preparation of 3α-p-toluenesulfonyloxy-7α,12α-dihydroxy-5β-cholan-24-carboxylic acid (Compound II-b)

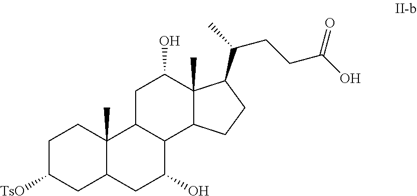

30 g (73.5 mmol) of cholic acid was dissolved in 100 ml of pyridine, cooled to 0° C., and 16.8 ml of p-toluenesulfonyl chloride (88.2 mmol) was then added thereinto. The mixture was continuously stirred for 2 h at 0 to 10° C. and poured into a mixture of 100 ml of ethyl acetate and 100 ml of water with stirring. Kept the mixture standing to separate out an organic layer. The water layer was washed with 100 ml of ethyl acetate for one time. Then the combined organic phase was washed with 50 ml of saturated salt solution, dried, and concentrated under reduced pressure to give II-b as a white foam solid which can be used in the next step of reaction directly.

### Example 4

### Preparation of 3β-azido-7α,12α-dihydroxy-5β-cholan-24-carboxylic acid (Compound III)

48.6 g (100 mmol) of II-a was dissolved in 150 ml of N,N-dimethyl formamide (DMF), and 26 g (400 mmol) of sodium azide and 21.2 g (400 mmol) ammonium chloride were then added thereinto. The reaction mixture was continuously stirred for 4 h at 90° C., and TLC was used to show that the reaction was complete. The reaction mixture was cooled to 40° C., added into 200 g of ice and filtered to afford III as an off-white solid.

III: ¹H NMR (300 MHz, DMSO-d₆): δ4.15(s, 2H), 3.99(s, 1H), 3.79(s, 1H), 3.63(s, 1H), 3.17(s, 2H), 2.63(m, 1H), 2.23(m, 1H), 2.14(m, 2H), 1.97(m, 1H), 1.62˜1.86(m, 6H), 0.91˜1.49(m, 16H), 0.86(s, 3H), 0.59(s, 3H); ESI-MS m/z (M−1)⁻432.

### Example 5

### Preparation of 3β-azido-7α,12α-dihydroxy-5β-cholan-24-carboxylic acid (Compound III)

48.6 g (100 mmol) of II-a was dissolved in 150 ml of acetonitrile, and 32.4 g (400 mmol) of potassium azide and 52.8 g (400 mmol) ammonium sulphate were then added thereinto. The reaction mixture was continuously stirred for 4 h at 80° C., and TLC was used to show that the reaction was complete. The reaction mixture was cooled to 40° C., added into 200 g of ice and filtered to give III as an off-white solid.

¹H NMR (300 MHz, DMSO-d₆): δ4.15(s, 2H), 3.99(s, 1H), 3.79(s, 1H), 3.63(s, 1H), 3.17(s, 2H), 2.63(m, 1H), 2.23(m, 1H), 2.14(m, 2H), 1.97(m, 1H), 1.62˜1.86 (m, 6H), 0.91˜1.49 (m, 16H), 0.86 (s, 3H), 0.59 (s, 3H); ESI-MS m/z (M−1)⁻432.

### Example 6

### Preparation of 3β-amino-7α,12α-dihydroxy-5β-cholan-24-carboxylic acid (Compound IV)

43.3 g (100 mmol) of III was dissolved in 250 ml of methanol, added with 4 g of 10% Pd—C and fed with hydrogen gas. The reaction mixture was continuously stirred for 16 h at 20° C., and TLC was used to show that the reaction was complete. The reaction mixture was filtered, concentrated under reduced pressure so as to remove methanol to afford a crude product. The crude product was recrystallized from ethyl acetate to give 30 g of product IV which was used in the next step of reaction directly.

¹H NMR (400 MHz, CD₃OD/D₂O=1/1): δ3.59 (s, 1H), 3.26 (s, 1H) 2.00˜0.75 (m, 30H), 0.52 (s, 3H); ESI-MS m/z (M+1) 408.

### Example 7

### Preparation of 3β-amino-7α,12α-dihydroxy-5β-cholan-24-carboxylic acid (Compound IV)

43.3 g (100 mmol) of III was dissolved in 180 ml of tetrahydrofuran, and added with 34 g (130 mmol) of triphenylphosphine and 10 ml of water. The reaction mixture was heated to be refluxed for 16 h, and TLC was used to show that the reaction was complete. The reaction mixture was cooled to room temperature and filtered, and the filtered cake was washed with ethyl acetate and dried to afford IV as a white solid which can be used in the next step of reaction directly.

¹H NMR (400 MHz, CD₃OD/D₂O=1/1): δ3.59 (s, 1H), 3.26 (s, 1H) 2.00˜0.75 (m, 30H), 0.52 (s, 3H); ESI-MS m/z (M+1) 408.

### Example 8

### Preparation of 3β-arachidylamido-7α,12α-dihydroxy-5β- cholan-24-carboxylic acid (Compound V, Aramchol)

41 g (100 mmol) of IV was dissolved in 1000 ml of dichloromethane, cooled to −20° C., and 30.3g (300 mmol) of triethylamine and 33 g (100 mmol) of arachidyl chloride were then added thereinto. The reaction mixture was continuously stirred for 16 h at −20° C. and poured into a mixture of 100 ml of ethyl acetate and 100 ml of ice water with stirring. Kept the mixture standing to separate out an organic layer. The water layer was washed with 100 ml of ethyl acetate for one time. Then the combined organic phase was washed with 50 ml of saturated salt solution, dried, and concentrated under reduced pressure to give a crude product of V. The crude product was recrystallized from acetone to afford 56 g of Compound IV (yield: 80%).

¹H NMR (300 MHz, DMSO-d₆): δ11.92(s, 1H), 7.51(d, J=6.3 Hz, 1H), 4.12(s, 1H), 4.04(s, 1H), 3.83(s, 1H), 3.77(s, 1H), 3.61(s, 1H), 0.82˜2.22(m, 69H), 0.56(s, 3H); ESI-MS m/z (M+1) 703.

1. A method for preparing
3β-arachidylamido-7α,12α,5β-cholan-24-carboxylic acid V, wherein V is prepared from Compound IV by the following reaction:

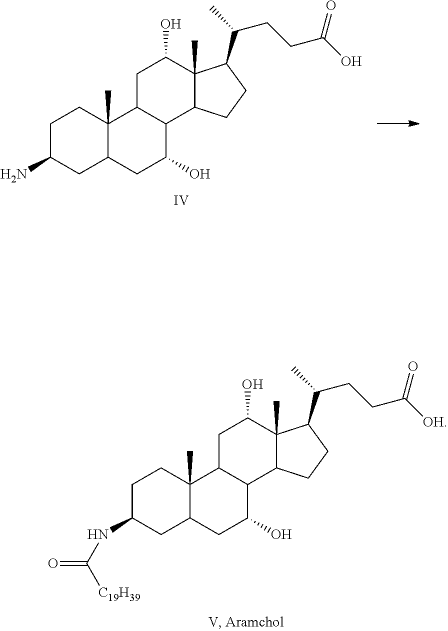

2. The method for preparing
3β-arachidylamido-7α,12α,5β-cholan-24-carboxylic acid as set forth in claim 1, wherein Compound IV is prepared from Compound III by the following reduction reaction:

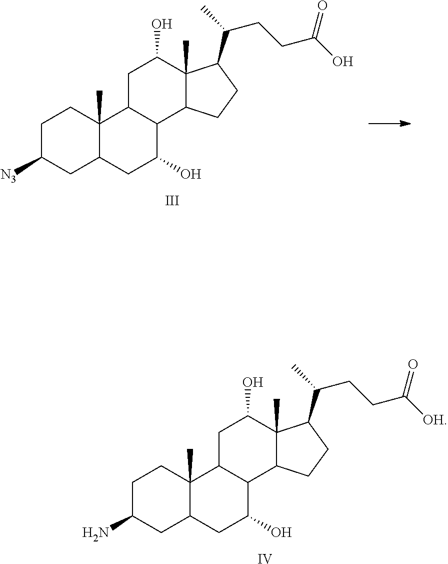

3. The method for preparing
3β-arachidylamido-7α,12α,5β-cholan-24-carboxylic acid as set forth in claim 2, wherein Compound III is prepared from Compound II and aziding agent by the following azidation reaction:

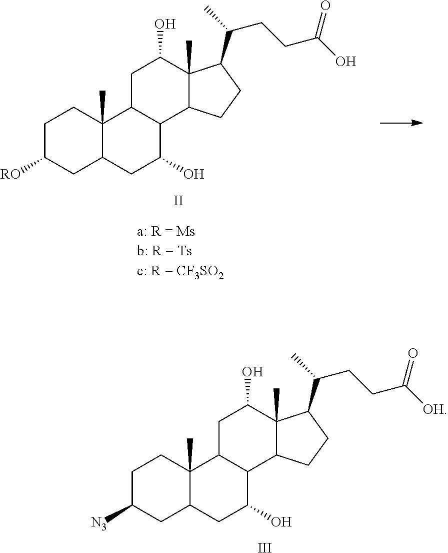

4. The method for preparing
3β-arachidylamido-7α,12α,5β-cholan-24-carboxylic acid as set forth in claim 3, wherein Compound II is prepared from cholic acid I by the following acylation reaction:

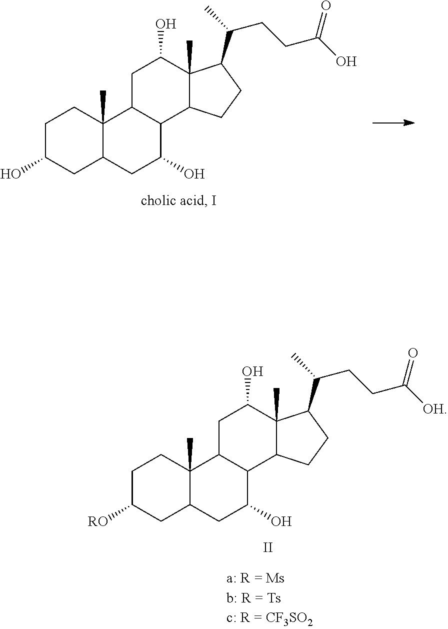

5. The method for preparing
3β-arachidylamido-7α,12α,5β-cholan-24-carboxylic acid as set forth in claim 1, wherein Compound V is obtained by the acylation of Compound IV and arachidyl chloride in the presence of a base, wherein the solvent used in the acylation is dichloromethane, chloroform, 1,2-dichloroethane, tetrahydrofuran, 1,4-dioxane, toluene, acetonitrile, ethyl acetate, pyridine, DMF or dimethoxyethane ; the said base is potassium carbonate, sodium carbonate, triethylamine, pyridine, N,N-dimethyl-4-aminopyridine, diisopropylethylamine or imidazole; and the mole ratio of Compound IV to arachidyl chloride is 1:0.8 to 1:3.

6. The method for preparing
3β-arachidylamido-7α,12α,5β-cholan-24-carboxylic acid as set forth in claim 2, wherein the solvent used in reduction reaction of Compound III is methanol, ethanol, isopropyl alcohol, tetrahydrofuran, dimethoxyethane, 1,4-dioxane,
N,N-dimethylformamide, N,N-dimethylacetamide, acetonitrile or water, or a mixture thereof; the catalyst used in the reaction is Pd—C, active nickel, cobalt chloride, nickel chloride or iron chloride; the reductant used in the reaction is hydrogen gas, sodium borohydride, potassium borohydride, lithium borohydride, hydrogen sulfide or triphenylphosphine.

7. The method for preparing
3β-arachidylamido-7α,12α,5β-cholan-24-carboxylic acid as set forth in claim 3, wherein the azidation reaction of Compound II and aziding agent is performed in an acidic condition; the used solvent is a polar solvent selected from
N,N-dimethylformamide, N,N-dimethylacetamide, acetonitrile, tetrahydrofuran, 1,4-dioxane, toluene, ethyl acetate and dimethoxyethane; the acidic agent used for the acidic condition is ammonium chloride, ammonium sulphate or ammonium bisulfate; the aziding agent is sodium azide, potassium azide or trimethylsilyl azide.

8. The method for preparing
3β-arachidylamido-7α,12α,5β-cholan-24-carboxylic acid as set forth in claim 4, wherein Compound II is obtained by performing the acylation reaction of cholic acid I and RCI in a basic condition; wherein, group R is mesyl Ms, p-tosyl Ts or trifyl CF₃SO₂; the used solvent is a non-protonic solvent selected from dichloromethane, chloroform, 1,2-dichloroethane, tetrahydrofuran, 1,4-dioxane, toluene, acetonitrile, ethyl acetate, pyridine, DMF, and dimethoxyethane; the used base is potassium carbonate, sodium carbonate, triethylamine, pyridine, N,N-dimethyl-4-aminopyridine, diisopropylethylamine or imidazole; the mole ratio of cholic acid to p-toluene sulfonyl chloride or methylsulfonyl chloride or trifluoromethanesulfonic anhydride or trifluoromathanesulfonylchloride is 1:0.8 to 1:3, 1:0.8 to 1:3.

9. The method for preparing
3β-arachidylamido-7α,12α,5β-cholan-24-carboxylic acid as set forth in claim 4, wherein Compound II has a structure of following formula:

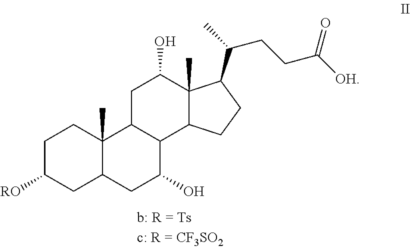

10. The method for preparing
3β-arachidylamido-7α,12α,5β-cholan-24-carboxylic acid as set forth in claim 1, wherein Compound V is obtained by the acylation of Compound IV and arachidyl chloride in the presence of a base, wherein the solvent used in the acylation is dichloromethane, chloroform, 1,2-dichloroethane, tetrahydrofuran, 1,4-dioxane, toluene, acetonitrile, ethyl acetate, pyridine, DMF or dimethoxyethane; the said base is potassium carbonate, sodium carbonate, triethylamine, pyridine, N,N-dimethyl-4-aminopyridine, diisopropylethylamine or imidazole; and the mole ratio of Compound IV to arachidyl chloride is 1:1 to 1:1.5.

11. The method for preparing
3β-arachidylamido-7α,12α,5β-cholan-24-carboxylic acid as set forth in claim 4, wherein Compound II is obtained by performing the acylation reaction of cholic acid I and RCI in a basic condition; wherein, group R is mesyl Ms, p-tosyl Ts or trifyl CF₃SO₂; the used solvent is a non-protonic solvent selected from dichloromethane, chloroform, 1,2-dichloroethane, tetrahydrofuran, 1,4-dioxane, toluene, acetonitrile, ethyl acetate, pyridine, DMF, and dimethoxyethane; the used base is potassium carbonate, sodium carbonate, triethylamine, pyridine, N,N-dimethyl-4-aminopyridine, diisopropylethylamine or imidazole;
the mole ratio of cholic acid to p-toluene sulfonyl chloride or methylsulfonyl chloride or trifluoromethanesulfonic anhydride or trifluoromathanesulfonylchloride is 1:1 to 1:1.5.
